(19) United States
(12) Patent Application Publication
(10) Pub. No.: US 2024/0197599 A1
(43) Pub. Date: Jun. 20, 2024

(54) COMPOSITIONS AND METHODS FOR REMOVING COLOR FROM HAIR

(71) Applicant: L'Oreal, Paris (FR)

(72) Inventors: Sarah SPEER, Linden, NJ (US); Shahid NASEER, Hasbrouck Heights, NJ (US)

(21) Appl. No.: 18/084,402

(22) Filed: Dec. 19, 2022

(51) Int. Cl. A61K 8/46; A61K 8/23; A61K 8/73; A61Q 5/08

(52) U.S. Cl. CPC A61K 8/46; A61K 8/23; A61K 8/738; A61Q 5/08

(57) ABSTRACT

The disclosure relates to compositions and methods for removing color from keratin fibers such as hair. The compositions comprise (a) at least one reducing agent, (b) at least one cyclodextrin compound, and (c) at least one solvent.

### TECHNICAL FIELD

The present disclosure relates to compositions and methods for removing color from keratin fibers, such as hair.

### BACKGROUND

Consumers often desire to change the color of their hair. For example, as a person ages, the hair follicle loses its natural pigment, resulting in grey, silver, or white hair. It is common for consumers to color their grey hairs, for example to match their natural hair color. Similarly, consumers may wish to change the color of their hair from that of their natural color. For example, consumers often wish to lighten or darken their hair color.

The process of altering the color of hair is complex, however, particularly when permanent (oxidative) hair dyes are used. In some cases, the process requires that the natural pigment (melanin) in the hair fiber be destroyed before dye molecules of the desired color are added. This process is typically used when an individual desires to change the color of their hair to a shade lighter than their natural color, by dissolving the melanin with an oxidizing agent before the oxidative dye molecules penetrate the hair fiber where they achieve the target shade by undergoing a chemical reaction with an oxidizing agent. If an individual wishes to change the color of their hair to a shade darker than their natural shade it may not be necessary to destroy the melanin in the hair fiber, but the oxidative dye must still undergo a reaction with an oxidizing agent to achieve the target shade.

However, if an individual who has previously changed the color of their hair with a first oxidative dye wishes to alter the color of their previously-colored hair to achieve a second color or shade, the first oxidative dye, which is not able to be washed out, must be neutralized so that it no longer imparts the first color to the hair fiber. Although there are known methods for removing the first color from the hair by neutralizing (reducing) the first oxidative dye molecules, these methods are not completely satisfactory because the first oxidative dye molecules can re-oxidize, thus interfering with the ability of the consumer to achieve the desired second color or shade. Other known methods for removing the first color from the hair use oxidizing agents, but these compositions are harsh and lead to damage to the hair causing the hair to be weak, brittle, and have an unhealthy appearance.

It has now been surprisingly discovered that a synergistic combination of components unexpectedly removes oxidative dyes from hair in a manner that also prevents the dyes from re-oxidizing. The combination of components therefore can be used to alter the overall color of the hair by removing all or substantially all of the previously-applied color, or to remove only certain colors or shades, in order to vary the color or tone of the hair without removing all color (e.g. to alter the color of the hair to a warmer or cooler tone).

The combination of components has the additional surprising benefit that the hair color removing compositions do not have the traditional strong odor associated with typical color-removing compositions, which odor most users find offensive.

### SUMMARY

The disclosure relates to compositions and methods that successfully remove color from the hair, in particular hair that has previously been dyed with an oxidative dye composition.

In various embodiments, the disclosure relates to compositions for treating keratin fibers such as hair, the compositions comprising (a) at least one reducing agent; (b) at least one cyclodextrin compound; and (c) at least one solvent. In various embodiments, reducing agents that may be chosen include but are not limited to cysteine and/or salts thereof, homocysteine and/or salts thereof, thiolactic acid and/or salts thereof, thioglycolic acid and/or esters thereof, borohydrides and/or derivatives thereof, phosphines and/or salts thereof, bisulphites and/or salts thereof, sulphites and/or salts thereof, or combinations of two or more thereof. Optionally, the reducing agent may comprise, consist essentially of, or consist of sulfur- and/or thio-based reducing agents and/or salts thereof. For example, in some embodiments, the reducing agent may be chosen from sulfites, bisulfites, and/or salts thereof, for example sodium sulfite, potassium sulfite, ammonium sulfite, sodium bisulfite, ammonium bisulfite, sodium metabisulfite, potassium metabisulfite, may be chosen from thio-based reducing agents such as thiolactic acid or thioglycolic acid, or may be chosen from combinations of two or more thereof. In various embodiments, the at least one cyclodextrin compound may be chosen from α-cyclodextrin, β-cyclodextrin, γ-cyclodextrin, methyl-α-cyclodextrin, methyl-β-cyclodextrin, methyl-γ-cyclodextrin, or combinations of two or more thereof. For example, in some embodiments, the cyclodextrin compound comprises, consists essentially of, or consists of β-cyclodextrin. The solvent may be chosen from water and/or non-aqueous solvents.

In various embodiments, the total amount of reducing agents in the composition can range from about 0.1% to about 20%, such as from about 1% to about 18%, from 2% to about 15%, or from about 4% to about 12% by weight, relative to the total weight of the composition. In some embodiments, the total amount of reducing agents ranges from about 3% to about 7%, from about 4% to about 6%, or is about 5% by weight, relative to the total weight of the composition. In other embodiments, the total amount of reducing agents ranges from about 8% to about 12%, from about 9% to about 11%, or is about 10% by weight, relative to the total weight of the composition.

In various embodiments, the total amount of cyclodextrin compounds in the composition can range from about 0.1% to about 20%, such as from about 1% to about 18%, from 2% to about 15%, or from about 4% to about 12% by weight, relative to the total weight of the composition. In some embodiments, the total amount of cyclodextrin compounds ranges from about 3% to about 7%, from about 4% to about 6%, or is about 5% by weight, relative to the total weight of the composition. In other embodiments, the total amount of cyclodextrin compounds ranges from about 8% to about 12%, from about 9% to about 11%, or is about 10% by weight, relative to the total weight of the composition.

In some embodiments, the compositions comprise total amounts of reducing agent(s) and cyclodextrin compound(s) such that the weight ratio of reducing agents to cyclodextrin compounds ranges from about 1 to about 10, such as from about 1 to about 5, from about 1 to about 3, or from about 1 to about 2.5.

Optionally, the compositions may comprise one or more additional components, such as carboxylic acids and/or salts thereof, surfactants, thickening agents, fatty compounds, or the like. In various embodiments, the compositions are free or essentially free of various components such as cationic surfactants, persulfates, peroxides, and/or ammonia.

The compositions are typically acidic, for example may have a pH ranging from about 1 to about 6, such as from about 2 to about 5, from about 2.5 to about 5, from about 3 to about 4.5, or from about 3.25 to about 4.25.

In exemplary embodiments, the disclosure relates to compositions for removing color from hair, the compositions comprising (a) at least one reducing agent chosen from sulfur-based reducing agents, thio-based reducing agents, salts thereof, or combinations thereof; (b) at least one cyclodextrin compound; and (c) at least one solvent. In various embodiments, the compositions comprise at least one reducing agent chosen from sulfites, bisulfites, salts thereof, or combinations thereof, for example chosen from sodium sulfite, potassium sulfite, ammonium sulfite, sodium bisulfite, ammonium bisulfite, sodium metabisulfite, and/or potassium metabisulfite, and/or at least one reducing agent chosen from thiolactic acid or thioglycolic acid. In various embodiments, the at least one cyclodextrin compound may be chosen from α-cyclodextrin, β-cyclodextrin, γ-cyclodextrin, methyl-α-cyclodextrin, methyl-β-cyclodextrin, methyl-γ-cyclodextrin, or combinations of two or more thereof, for example may comprise, consist essentially of, or may consist of β-cyclodextrin. Optionally, the total amount of reducing agents and/or cyclodextrin compounds may independently range from about 1% to about 15%, and in various embodiments the composition may have a weight ratio of the total amount of reducing agents to the total amount of cyclodextrin compounds ranging from about 1 to about 10, such as from about 1 to about 5, or from about 1 to about 3. The compositions may comprise additional components such as anionic surfactants, non-ionic surfactants, amphoteric/zwitterionic surfactants, carboxylic acids and/or salts thereof, thickening agents, fatty compounds, or combinations of two or more thereof.

In further exemplary embodiments, the disclosure relates to compositions for removing color from hair, the compositions comprising (a) at least one reducing agent; (b) at least one cyclodextrin compound; and (c) at least one solvent, wherein the composition comprises total amounts of reducing agent(s) and cyclodextrin compound(s) such that the weight ratio of reducing agents to cyclodextrin compounds ranges from about 1 to about 5, such as from about 1 to about 3, or from about 1 to about 2.5. In various embodiments, the compositions comprise at least one reducing agent chosen from sulfites, bisulfites, salts thereof, or combinations thereof, for example chosen from sodium sulfite, potassium sulfite, ammonium sulfite, sodium bisulfite, ammonium bisulfite, sodium metabisulfite, and/or potassium metabisulfite, and/or at least one reducing agent chosen from thiolactic acid or thioglycolic acid. In various embodiments, the at least one cyclodextrin compound may be chosen from α-cyclodextrin, β-cyclodextrin, γ-cyclodextrin, methyl-α-cyclodextrin, methyl-β-cyclodextrin, methyl-γ-cyclodextrin, or combinations of two or more thereof, for example may comprise, consist essentially of, or may consist of β-cyclodextrin. Optionally, the total amount of reducing agents may range from about 0.1% to about 20%, and the total amount of cyclodextrin compounds may range from about 0.1% to about 20%. The compositions may comprise additional components such as anionic surfactants, non-ionic surfactants, amphoteric/zwitterionic surfactants, carboxylic acids and/or salts thereof, thickening agents, fatty compounds, or combinations of two or more thereof.

The disclosure also relates to methods of treating hair using the compositions according to the disclosure. For example, the methods may be methods of removing oxidative dyes from the hair. In some embodiments, a composition according to the disclosure may be applied to the hair and left on the hair for a period of time, for example up to about 2 hours, such as from about 30 seconds to about 2 hours, from about 1 minute to about 1 hour, from about 1 minute to about 45 minutes, from about 1 minute to about 30 minutes, from about 5 minutes to about 1 hour, from about 5 minutes to about 45 minutes, from about 5 minutes to about 30 minutes, etc. The methods may further comprise rinsing the hair.

### BRIEF DESCRIPTION OF THE FIGURE

The patent or application file contains at least one drawing executed in color. Copies of this patent or patent application publication with color drawing(s) will be provided by the Office upon request and payment of the necessary fee.

FIG. 1 shows images of two identical swatches of hair colored with the same oxidative hair dye composition and subsequently treated with either a hair color-removing composition according to the disclosure (1B) or a comparative hair color-removing composition (C3). The swatch treated with composition 1B is warmer, with more yellow undertones and less blue undertones, whereas the swatch treated with composition C3 is cooler, with more blue undertones.

### DETAILED DESCRIPTION

The disclosure relates to compositions and methods for removing color, in particular color imparted by previously-applied oxidative dyes, from the hair. The color-removal benefits according to various embodiments may be long-lasting, e.g. may last for at least 7 days, at least 1 month, at least 2 months, etc.

### I. Compositions

The compositions according to the disclosure comprise (a) at least one reducing agent, (b) at least one cyclodextrin compound, and (c) at least one solvent.

### Reducing Agents

Compositions according to the disclosure comprise at least one reducing agent. By way of example, useful reducing agents may be chosen from cysteine and/or salts thereof, homocysteine and/or salts thereof, thiolactic acid and/or salts thereof, thioglycolic acid and/or esters thereof, borohydrides and/or derivatives thereof, phosphines and/or salts thereof, bisulphites and/or salts thereof, sulphites and/or salts thereof, or combinations of two or more thereof. Non-limiting examples of salts of the foregoing include sodium salts, ammonium salts, lithium salts, potassium salts, and calcium salts. In various embodiments, sulfur-based (e.g. sulfites, bisulfites, metabisulfites) and/or thio-based (e.g. thiolactic acid, thioglycolic acid) reducing agents and/or salts thereof may be used.

In various embodiments, the at least one reducing agent is chosen from sulfites, bisulfites, and/or salts thereof. For example, the at least one reducing agent may be chosen from sodium sulfite, potassium sulfite, ammonium sulfite, sodium bisulfite, ammonium bisulfite, sodium metabisulfite, potassium metabisulfite, or combinations of two or more thereof. In some embodiments, the at least one reducing agent comprises, consists essentially of, or consists of sodium metabisulfite, potassium metabisulfite, or a combination thereof. In certain embodiments, the at least one reducing agent comprises sodium metabisulfite. In other embodiments, the reducing agent is chosen from thioglycolic acid, thiolactic acid, or a combination thereof. In still further embodiments, the reducing agent comprises a combination of at least two of any of the foregoing.

In various embodiments, the total amount of reducing agents may range from about 0.1% to about 20%, such as from about 1% to about 18%, from 2% to about 15%, or from about 4% to about 12% by weight, relative to the total weight of the composition. For example, in various embodiments, the total amount of reducing agents may range from about 1% to about 20%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 2% to about 20%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 3% to about 20%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 4% to about 20%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 25%, from about 5% to about 20%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6%, from about 6% to about 20%, from about 6% to about 15%, from about 6% to about 14%, from about 6% to about 13%, from about 6% to about 12%, from about 6% to about 11%, from about 6% to about 10%, from about 6% to about 9%, from about 6% to about 8%, from about 6% to about 7%, from about 7% to about 20%, from about 7% to about 15%, from about 7% to about 14%, from about 7% to about 13%, from about 7% to about 12%, from about 7% to about 11%, from about 7% to about 10%, from about 7% to about 9%, from about 7% to about 8%, from about 8% to about 20%, from about 8% to about 15%, from about 8% to about 14%, from about 8% to about 13%, from about 8% to about 12%, from about 8% to about 11%, from about 8% to about 10%, from about 8% to about 9%, from about 9% to about 20%, from about 9% to about 15%, from about 9% to about 14%, from about 9% to about 13%, from about 9% to about 12%, from about 9% to about 11%, from about 9% to about 10%, from about 10% to about 20%, from about 10% to about 15%, from about 10% to about 14%, from about 10% to about 13%, from about 10% to about 12%, or from about 10% to about 11% by weight, relative to the total weight of the composition. For example, the total amount of reducing agents may be about 3%, about 4%, about 5%, about 6%, about 7%, about 8%, about 9%, about 10%, about 11%, or about 12% by weight, relative to the total weight of the composition, including any range using any of the foregoing as upper and lower limits.

As a non-limiting example, the reducing agent may comprise, consist essentially of, or consist of sulfur- and/or thio-based reducing agents and/or salts thereof, and the total amount of reducing agents may be about 2%, about 3%, about 4%, about 5%, about 6%, about 7%, about 8%, about 9%, about 10%, about 11%, about 12%, about 13%, about 14%, or about 15% by weight, relative to the total weight of the composition, or may be a range using any of the foregoing as upper or lower limits.

As a further non-limiting example, the reducing agent may comprise, consist essentially of, or consist of thioglycolic acid, thiolactic acid, sulfites, bisulfites, metabisulfites, salts thereof, or combinations of two or more thereof, and the total amount of reducing agents may be about 2%, about 3%, about 4%, about 5%, about 6%, about 7%, about 8%, about 9%, about 10%, about 11%, about 12%, about 13%, about 14%, or about 15% by weight, relative to the total weight of the composition, or may be a range using any of the foregoing as upper or lower limits.

As yet a further non-limiting example, the reducing agent may comprise, consist essentially of, or consist of thioglycolic acid, thiolactic acid, sodium sulfite, potassium sulfite, ammonium sulfite, sodium bisulfite, ammonium bisulfite, sodium metabisulfite, potassium metabisulfite, or combinations of two or more thereof, and the total amount of reducing agents may be about 2%, about 3%, about 4%, about 5%, about 6%, about 7%, about 8%, about 9%, about 10%, about 11%, about 12%, about 13%, about 14%, or about 15% by weight, relative to the total weight of the composition, or may be a range using any of the foregoing as upper or lower limits.

### Cyclodextrin Compounds

Compositions according to the disclosure comprise at least one cyclodextrin compound. In some embodiments, the compositions comprise more than one cyclodextrin compound, for example, at least two cyclodextrin compounds, at least three cyclodextrin compounds, etc.

Cyclodextrins are a family of cyclic oligosaccharides consisting of a macrocyclic ring of glucose subunits joined by α-1,4 glycosidic bonds. The cyclodextrins that can be used include those of the formula:

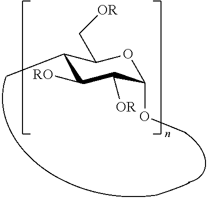

wherein:
R is chosen from H, CH3, or a hydroxypropyl group, and
n ranges from 6-8.

For example, in embodiments where R=H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R=CH3, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodextrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

The total amount of cyclodextrin compounds may range from about 0.1% to about 20%, such as from about 1% to about 18%, from 2% to about 15%, or from about 4% to about 12% by weight, relative to the total weight of the composition. For example, in various embodiments, the total amount of cyclodextrin compounds may range from about 1% to about 20%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 2% to about 20%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 3% to about 20%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 4% to about 20%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 25%, from about 5% to about 20%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6%, from about 6% to about 20%, from about 6% to about 15%, from about 6% to about 14%, from about 6% to about 13%, from about 6% to about 12%, from about 6% to about 11%, from about 6% to about 10%, from about 6% to about 9%, from about 6% to about 8%, from about 6% to about 7%, from about 7% to about 20%, from about 7% to about 15%, from about 7% to about 14%, from about 7% to about 13%, from about 7% to about 12%, from about 7% to about 11%, from about 7% to about 10%, from about 7% to about 9%, from about 7% to about 8%, from about 8% to about 20%, from about 8% to about 15%, from about 8% to about 14%, from about 8% to about 13%, from about 8% to about 12%, from about 8% to about 11%, from about 8% to about 10%, from about 8% to about 9%, from about 9% to about 20%, from about 9% to about 15%, from about 9% to about 14%, from about 9% to about 13%, from about 9% to about 12%, from about 9% to about 11%, from about 9% to about 10%, from about 10% to about 20%, from about 10% to about 15%, from about 10% to about 14%, from about 10% to about 13%, from about 10% to about 12%, or from about 10% to about 11% by weight, relative to the total weight of the composition. For example, the total amount of cyclodextrin compounds may be about 3%, about 4%, about 5%, about 6%, about 7%, about 8%, about 9%, about 10%, about 11%, or about 12% by weight, relative to the total weight of the composition, including any range using any of the foregoing as upper and lower limits.

Optionally, in at least some embodiments, it may be advantageous to choose amounts of reducing agent(s) and cyclodextrin compound(s) such that the weight ratio of reducing agents to cyclodextrin compounds is at least about 1, for example is greater than 1. By way of example only, in various embodiments the weight ratio of the total amount of reducing agents to the total amount of cyclodextrin compounds may range from about 1 to about 10, from about 1.25 to about 8, from about 1.5 to about 6, from about 1.75 to about 4, from about 1.8 to about 3.5, from about 1.9 to about 3, or from about 2 to about 2.5. As further examples, the weight ratio of the total amount of reducing agents to the total amount of cyclodextrin compounds may range from about 1 to about 5, from about 1 to about 4.5, from about 1 to about 4, from about 1 to about 3.5, from about 1 to about 3, from about 1 to about 2.5, from about 1 to about 2.25, from about 1 to about 2, from about 1 to about 1.75, from about 1 to about 1.5, or from about 1 to about 1.25.

### Solvents

Compositions according to the disclosure comprise at least one solvent. The solvent may be chosen from water, non-aqueous solvents, or combinations thereof.

In some embodiments, the solvent comprises, consists essentially of, or consists of water. The total amount of water may vary depending on the desired properties of composition, for example consistency, viscosity, etc.

In some embodiments, non-aqueous solvents maybe used, for example, glycerin, C₁₋₄ alcohols, fatty alcohols, fatty ethers, fatty esters, polyols, glycols, vegetable oils, mineral oils, liposomes, laminar lipid materials, or combinations thereof. Non-limiting examples of non-aqueous solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

The total amount of solvents in the composition typically ranges from about 50% to about 98%, such as from about 60% to about 98%, from about 70% to about 98%, from about 75% to about 98%, from about 75% to about 95%, or from about 80% to about 90% by weight, relative to the total weight of the composition.

### Surfactants

Compositions according to the disclosure may optionally comprise at least one surfactant chosen from anionic, non-ionic, and/or amphoteric or zwitterionic surfactants. For example, the compositions may comprise one or more anionic surfactants, one or more non-ionic surfactants, or one or more amphoteric/zwitterionic surfactants, or the compositions may comprise mixtures of surfactants having the same or different ionicities.

### Anionic Surfactants

In at least some embodiments, the compositions comprise at least at least one anionic surfactant. The term “anionic surfactant” means a surfactant comprising, as ionic or ionizable groups, only anionic groups. A species is termed as being “anionic” when it bears at least one permanent negative charge or when it can be ionized as a negatively charged species, under the conditions of use of the composition (for example the medium or the pH) and not comprising any cationic charge. These anionic groups may be chosen from —CO₂H, —CO₂⁻, —SO₃H, —SO₃⁻, —OSO₃H, —OSO₃⁻, —H₂PO₃, —HPO₃⁻, —PO₃²⁻, —H₂PO₂, ═HPO₂, —HPO₂⁻, ═PO₂⁻, ═POH, and ═PO⁻ groups.

The anionic surfactants may be sulfate, sulfonate, and/or carboxylic (or carboxylate) surfactants, or mixtures thereof.

Sulfate anionic surfactants comprise at least one sulfate function but do not comprise any carboxylate or sulfonate functions. The sulfate anionic surfactants that may be used at least one sulfate comprise function (—OSO₃H or —OSO₃).

Non-limiting examples of sulfate anionic surfactants include alkyl sulfates, alkyl ether sulfates, alkylamido ether sulfates, alkylaryl polyether sulfates, monoglyceride sulfates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

Sulfonate anionic surfactants comprise at least one sulfonate function (—SO₃H or —SO₃⁻) and may optionally also comprise one or more sulfate functions, but do not comprise any carboxylate functions. The sulfonate anionic surfactants that may be used comprise at least one sulfonate function (—SO₃H or —SO₃⁻).

Non-limiting examples of sulfonate anionic surfactants include alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

Carboxylate anionic surfactants comprise at least one carboxylic or carboxylate function (—COOH or —COO⁻) and may optionally also comprise one or more sulfate and/or sulfonate functions. The carboxylic anionic surfactants that may be used thus comprise at least one carboxylic or carboxylate function (—COOH or —COO⁻).

Non-limiting examples of carboxylate anionic surfactants include acylglycinates, acyllactylates, acylsarcosinates, acylglutamates, alkyl-D-galactosideuronic acids, alkyl ether carboxylic acids, alkyl(C6-30 aryl) ether carboxylic acids, alkylamido ether carboxylic acids; and also the salts of these compounds; the alkyl and/or acyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

When the anionic surfactant is in salt form, the salt may be chosen from alkali metal salts, such as the sodium or potassium salt, ammonium salts, amine salts and in particular amino alcohol salts, and alkaline-earth metal salts, such as the magnesium salt.

Examples of amino alcohol salts may include but are not limited to monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

In some embodiments, alkali metal or alkaline-earth metal salts and in particular the sodium or magnesium salts may be chosen, especially in the form of alkali metal, ammonium, amino alcohol and alkaline-earth metal salts, or a mixture of these compounds, may be chosen.

In exemplary and non-limiting embodiments, the anionic surfactant may be chosen from sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C14-16 Olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, sodium cocoyl glutamate, disodium cocoyl glutamate, potassium myristoyl glutamate, TEA-cocoyl glutamate, sodium cocoyl glycinate, potassium cocoyl glycinate, sodium cocoyl alaninate, TEA-cocoyl alaninate, or a combination of two or more thereof. For example, the compositions may comprise at least one anionic surfactant chosen from sodium laureth sulfate, sodium lauryl sulfate, sodium lauroyl sulfate, sodium lauroyl methyl isethionate, or a combination of two or more thereof.

If present, the total amount of anionic surfactants may range up to about 15%, such as up to about 12%, up to about 10%, up to about 8%, up to about 5%, up to about 3.5%, or up to about 2% by weight, relative to the total weight of the composition. For example, the total amount of anionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one anionic surfactant, and have a total amount of anionic surfactants ranging from about 0.25% to about 5%, such as from about 0.5% to about 4%, from about 0.75% to about 3%, or from about 1% to about 2% by weight, relative to the total weight of the composition.

### Amphoteric or Zwitterionic Surfactants

In at least some embodiments, the compositions comprise at least one amphoteric or zwitterionic surfactant. Non-limiting examples of useful amphoteric surfactants include derivatives of aliphatic secondary and tertiary amines where the aliphatic radical can be straight or branched chain and one of the aliphatic substituents contains from about 8 to about 18 carbon atoms and one contains an anionic group such as carboxy, sulfonate, sulfate, phosphate, or phosphonate. Exemplary amphoteric surfactants include sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, and lauryl diethylenediaminoglycine, as well as combinations of two or more thereof.

Betaines may also be used. For example, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, oleyl betaine, cocamidopropyl betaine, or combinations of two or more thereof, may be chosen.

If present, the total amount of amphoteric or zwitterionic surfactants may range up to about 15%, such as up to about 12%, up to about 10%, up to about 8%, up to about 5%, up to about 3.5%, or up to about 2% by weight, relative to the total weight of the composition. For example, the total amount of amphoteric or zwitterionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one amphoteric or zwitterionic surfactant, and have a total amount of amphoteric or zwitterionic surfactants ranging from about 0.25% to about 5%, such as from about 0.5% to about 4%, from about 0.75% to about 3%, or from about 1% to about 2% by weight, relative to the total weight of the composition. In at least some other embodiments, the compositions are free or substantially free of amphoteric surfactants.

### Nonionic Surfactants

The nonionic surfactants may be chosen from alcohols, α-diols and (C₁-C₂₀) alkylphenols, these compounds being polyethoxylated, polypropoxylated and/or polyglycerolated, the number of ethylene oxide and/or propylene oxide groups possibly ranging from 1 to 100, and the number of glycerol groups possibly ranging from 2 to 30, or alternatively these compounds comprising at least one fatty chain comprising from 8 to 30 carbon atoms and especially from 16 to 30 carbon atoms. For example, nonionic surfactants may be chosen from monooxyalkylenated or polyoxyalkylenated (C₈-C₂₄)alkylphenols, saturated or unsaturated, linear or branched, monooxyalkylenated or polyoxyalkylenated C₈-C₃₀ alcohols, saturated or unsaturated, linear or branched, monooxyalkylenated or polyoxyalkylenated C₈-C₃₀ amides, esters of saturated or unsaturated, linear or branched, C₈-C₃₀ acids and of polyalkylene glycols, monooxyalkylenated or polyoxyalkylenated esters of saturated or unsaturated, linear or branched, C₈-C₃₀ acids and of sorbitol, saturated or unsaturated, monooxyalkylenated or polyoxyalkylenated plant oils, condensates of ethylene oxide and/or of propylene oxide, or combinations thereof.

By way of example only, the adducts of ethylene oxide with lauryl alcohol, for example those containing from 9 to 50 oxyethylene units or from 10 to 12 oxyethylene units (Laureth-10 to Laureth-12); the adducts of ethylene oxide with behenyl alcohol, for example those containing from 9 to 50 oxyethylene units (Beheneth-9 to Beheneth-50); the adducts of ethylene oxide with cetearyl alcohol (mixture of cetyl alcohol and stearyl alcohol), for example those containing from 10 to 30 oxyethylene units (Ceteareth-10 to Ceteareth-30); the adducts of ethylene oxide with cetyl alcohol, for example those containing from 10 to 30 oxyethylene units (Ceteth-10 to Ceteth-30); the adducts of ethylene oxide with stearyl alcohol, for example those containing from 10 to 30 oxyethylene units (Steareth-10 to Steareth-30); the adducts of ethylene oxide with isostearyl alcohol, for example those containing from 10 to 50 oxyethylene units (Isosteareth-10 to Isosteareth-50); monoglycerolated or polyglycerolated C₈-C₄₀, e.g. C₈-C₃₀, alcohols, such as lauryl alcohol containing 4 mol of glycerol (Polyglyceryl-4 Lauryl Ether), lauryl alcohol containing 1.5 mol of glycerol, oleyl alcohol containing 4 mol of glycerol (Polyglyceryl-4 Oleyl Ether), oleyl alcohol containing 2 mol of glycerol (Polyglyceryl-2 Oleyl Ether), cetearyl alcohol containing 2 mol of glycerol, cetearyl alcohol containing 6 mol of glycerol, oleocetyl alcohol containing 6 mol of glycerol, or octadecanol containing 6 mol of glycerol; polyoxyethylenated fatty esters such as the adducts of ethylene oxide with esters of lauric acid, palmitic acid, stearic acid or behenic acid, and mixtures thereof, for example those containing from 9 to 100 oxyethylene units such as PEG-9 to PEG-50 laurate, PEG-9 to PEG-50 palmitate, PEG-9 to PEG-50 stearate, PEG-9 to PEG-50 palmitostearate, PEG-9 to PEG-50 behenate, polyethylene glycol 100 EO monostearate (PEG-100 stearate); glyceryl stearate (glyceryl mono-, di- and/or tristearate); glyceryl ricinoleate; sorbitan palmitate; sorbitan isostearate; sorbitan stearate; sorbitan palmitate; sorbitan trioleate; alkylglucose sesquistearates such as methylglucose sesquistearatel alkylglucose palmitates such as methylglucose or ethylglucose palmitate, etc., or combinations of two or more thereof may be chosen.

If present, the total amount of nonionic surfactants may range up to about 15%, such as up to about 12%, up to about 10%, up to about 8%, up to about 5%, up to about 3.5%, or up to about 2% by weight, relative to the total weight of the composition. For example, the total amount of nonionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one nonionic surfactant, and have a total amount of nonionic surfactants ranging from about 0.25% to about 5%, such as from about 0.5% to about 4%, from about 0.75% to about 3%, or from about 1% to about 2% by weight, relative to the total weight of the composition. In at least some embodiments, the compositions are free or substantially free of nonionic surfactants.

In some embodiments, compositions according to the disclosure are free or substantially free of cationic surfactants.

### Fatty Compounds

Optionally, compositions according to the disclosure may include at least one fatty compound. In certain embodiments, the at least one fatty compound may be chosen from lower alkanes, fatty alcohols, fatty acids, esters of fatty acids, esters of fatty alcohols, oils such as mineral, vegetable, animal, silicone and non-silicone oils, silicone and non-silicone waxes, or combinations of any two or more thereof. In some embodiments, the compositions comprise at least one fatty compound of natural origin. In some embodiments, the compositions are free of fatty compounds that are not of natural origin.

Non-limiting examples of silicone oils include dimethicone, amodimethicone, cyclomethicone, polysilicone-11, phenyl trimethicone, trimethylsilylamodimethicone, and stearoxytrimethylsilane. For example, the composition may comprise at least one silicone chosen from amodimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-17/18 Dimethicone, cetyl PEG/PPG-10/1 dimethicone, Dimethicone PEG-8 Benzoate, Dimethicone PEG-7 Phosphate, Dimethicone PEG-8 Phosphate, Dimethicone PEG-10 Phosphate, or a combination of two or more thereof. In some preferred embodiments, the compositions comprise a silicone oil component that comprises, consists essentially of, or consists of dimethicone, amodimethicone, or a combination thereof. In some embodiments, the compositions are free or substantially free of silicone oils and/or silicone waxes.

In some embodiments, compositions according to the disclosure may include at least one fatty compound chosen from fatty alcohols. In certain embodiments, “fatty alcohol” refers to any alcohol with a carbon chain of C5 or greater, such as, for example, C8 or greater, C10 or greater, or C12 or greater, such as from 6 to 30 carbon atoms or from 8 to 30 carbon atoms. The fatty alcohols may be alkoxylated or non-alkoxylated, saturated or unsaturated, and linear or branched. Non-limiting examples of fatty alcohols include arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, or combinations of two or more thereof. In some preferred embodiments, the compositions comprise a fatty alcohol component that comprises, consists essentially of, or consists of cetyl alcohol, stearyl alcohol, cetearyl alcohol, or combinations thereof.

In some embodiments, the composition may include one or more fatty compounds chosen from oils of animal, vegetable, or mineral origin (e.g. lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, or shea butter), linear or branched hydrocarbons (e.g. polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane), mono- and/or polyesters of fatty acids and/or of fatty alcohols (e.g. mono- and polyesters of hydroxy acids and of fatty alcohols, esters of benzoic acid and of fatty alcohols, polyesters of polyols, dipentaerythrityl C5-C9 esters, trimethylolpropane polyesters, propylene glycol polyesters, or polyesters of hydrogenated castor oil), perfluorinated and/or organofluorinated oils, fluorosilicone oils, or combinations of two or more thereof. Non-limiting examples of fatty acids include optionally branched and/or unsaturated fatty acids such as myristic acid, palmitic acid, stearic acid, behenic acid, oleic acid, linoleic acid, linolenic acid, isostearic acid, or combinations of two or more thereof.

If present, the total amount of fatty compounds in the composition may range up to about 20%, such as up to about 18%, up to about 15%, up to about 12%, up to about 10%, up to about 8%, or up to about 5% by weight, relative to the total weight of the composition. For example, the total amount of fatty compounds may range from about 0.1% to about 20%, such as from about 0.5% to about 18%, from about 1% to about 15%, from about 1.25% to about 12%, or from about 1.5% to about 10% by weight, relative to the total weight of the composition. In at least some embodiments, the compositions are free or essentially free of fatty compounds.

### Thickening Agents

Compositions according to the disclosure optionally comprise at least one thickening agent. Useful thickening agents include, but are not limited to, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, guar gum, petroleum jelly, waxes, particulate associate colloids, such as bentonite, colloidal silicon dioxide, and microcrystalline cellulose, celluloses such as hydroxyethylcellulose and hydroxypropylcellulose, and guars such as hydroxypropyl guar.

In some embodiments, the thickening agent may be chosen from associative thickening polymers such as anionic associative polymers, amphoteric associative polymers, cationic associative polymers, or nonionic associative polymers. A non-limiting example of an amphoteric associative polymer is acrylates/beheneth-25 methacrylate copolymer, and non-limiting examples of anionic associative polymers include acrylates copolymer and acrylates crosspolymer-4.

If present, the total amount of thickening agents may range from about 0.001% to about 5%, such as from about 0.01% to about 4%, from about 0.1% to about 3.5%, from about 0.2% to about 3%, from about 0.3% to about 2.5% by weight, from about 0.4% to about 2%, from about 0.5% to about 1.5%, or from about 0.5% to about 1%, relative to the total weight of the composition.

### Carboxylic Acids

Compositions according to the disclosure optionally comprise at least one carboxylic acid. In some embodiments, the compositions comprise more than one carboxylic acid, for example, at least two carboxylic acids, at least three carboxylic acids, etc. For example, mono-, di-, or tri-carboxylic acids may be chosen.

In various embodiments, the at least one carboxylic acid may be chosen from citric acid, maleic acid, succinic acid, aspartic acid, glutamic acid, lactic acid, malic acid, tartaric acid, salts thereof, or combinations thereof, preferably citric acid, lactic acid, salts thereof, or combinations thereof. In one embodiment, citric acid may be chosen.

If present, the total amount of carboxylic acids may range up to about 3%, such as up to about 2.5%, up to about 2%, up to about 1.5%, up to about 1%, or up to about 0.5% by weight, relative to the total weight of the composition. For example, the total amount of carboxylic acids may range from about 0.001% to about 3%, from about 0.01% to about 2.5%, from about 0.1% to about 2%, or from about 0.1% to about 1.5% by weight, relative to the total weight of the composition. In some embodiments, the total amount of mono-, di-, and tri-carboxylic acids does not exceed about 1%, for example may range from 0.001% to about 1%, such as from about 0.001% to about 0.9%, from about 0.001% to about 0.8%, from about 0.001% to about 0.7%, from about 0.001% to about 0.6%, from about 0.001% to about 0.5%, from about 0.001% to about 0.4%, from about 0.001% to about 0.3%, from about 0.001% to about 0.2%, or from about 0.001% to about 0.1% by weight, relative to the total weight of the composition.

In some embodiments, the compositions are free or essentially free of carboxylic acids, or are free or essentially free of mono-, di-, and/or tri-carboxylic acids. In other embodiments, the compositions are free or essentially free of citric acid.

### Auxiliary Components

Compositions according to the disclosure may optionally include one or more auxiliary components. Non-limiting examples include preservatives, fragrances such as parfum, pH adjusters (e.g. citric acid, maleic acid, malic acid, malonic acid, etc.), salts, antioxidants, vitamins (e.g. ascorbic acid, tocopherol), vitamin derivatives, amino acids (e.g. betaine, arginine, glycine, proline, etc.), amines (e.g. monoethanolamine, diethanolamine, triethanolamine, etc.), botanical extracts, buffers, sequestering agents, and the like. In some embodiments, the compositions are free or essentially free of any of the aforementioned, e.g. the compositions may be free or essentially free of antioxidants, free or essentially free of ascorbic acid, etc.

The total amount of auxiliary components, if present, typically ranges from about 0.01% to about 15% by weight, based on the total weight of the composition. For example, in some embodiments the individual amounts of each component or the total amount of components may range from about 0.1% to about 10%, about 0.1% to about 8%, about 0.1% to about 5%, about 0.1% to about 4%, about 0.1% to about 3%, about 0.1% to about 2%, about 0.25% to about 10%, about 0.25% to about 8%, about 0.25% to about 5%, about 0.25% to about 4%, about 0.25% to about 3%, about 0.25% to about 2%, about 0.5% to about 10%, about 0.5% to about 8%, about 0.5% to about 5%, about 0.5% to about 4%, about 0.5% to about 3%, about 0.5% to about 2%, about 0.75% to about 10%, about 0.75% to about 8%, about 0.75% to about 5%, about 0.75% to about 4%, about 0.75% to about 3%, or about 0.75% to about 2% by weight, based on the total weight of the composition.

In some embodiments, the compositions may be free of ascorbic acid, persulfates, peroxides, and/or ammonia. In yet other embodiments, the compositions may be essentially free of ascorbic acid, persulfates, peroxides, and/or ammonia, for example may comprise less than 5%, less than 4.5%, less than 4%, less than 3.5%, less than 3%, less than 2.5%, less than 2%, less than 1.5%, less than 1.25%, less than 1%, less than 0.75%, less than 0.5%, less than 0.4%, less than 0.3%, less than 0.2%, less than 0.1%, less than 0.05%, or less than 0.01% of a total amount of ascorbic acid, persulfates, peroxides, or ammonia, or of a total amount of any combination of two or more thereof.

The compositions may be in any suitable form. For example, the compositions may be a liquid, a gel, a gel cream, a cream, a serum, etc. Advantageously, the compositions can be used as a one-part hair color removing composition, as compared to known systems for removing color from hair that require the application of multiple compositions to effectively remove color from the hair.

The pH of the composition is typically acidic, i.e. below 7, such as below about 6, below about 5, or below about 4. For example, compositions according to the disclosure may have a pH ranging from about 1 to about 6, such as from about 2 to about 5. In some embodiments, the compositions have a pH ranging from about 2.5 to about 5, from about 3 to about 4.5, or from about 3.25 to about 4.25.

### II. Methods

The disclosure further relates to methods of removing color from keratin fibers, especially hair that has been previously dyed such as with an oxidation dye. The methods comprise applying a composition according to the disclosure onto the keratin fibers, e.g. hair, optionally leaving the composition on the keratin fibers for a period of time (“rest period,” “resting period,” or “leave-in period”), and subsequently rinsing the composition from the keratin fibers. Thus, the methods may comprise a two-step method, which is simpler and more effective than known three- or more step methods where multiple compositions are applied to the hair for color removal before the hair is rinsed.

The appropriate leave-in period will be determined based on the color to be removed, and may in various embodiments last up to about 1 minute, up to about 2 minutes, up to about 5 minutes, up to about 10 minutes, up to about 20 minutes, up to abut 30 minutes, up to about 45 minutes, up to about 1 hour, up to about 2 hours, etc., such as from about 1 minute to about 60 minutes, from about 2 minutes to about 45 minutes, from about 5 minutes to about 40 minutes, or from about 10 minutes to about 30 minutes.

Surprisingly, the compositions and methods according to the disclosure effectively remove color imparted to hair by previously-applied oxidative dyes without the use of persulfates, peroxides, and/or ammonia, and can prevent or substantially prevent the oxidative dyes from re-oxidizing such that the color does not return. Thus, in at least some embodiments, the compositions are free or essentially free of persulfates, peroxides, and/or ammonia, and/or the methods include only compositions that are free or substantially free of persulfates, peroxides, and/or ammonia. This benefit has surprisingly been observed on hair even several months after the original color was removed with compositions and methods according to the disclosure, and thus the color removal is considered to be permanent, which is a benefit not achieved by traditional hair color removing compositions that use reducing agents.

In addition, the synergistic combination of components in compositions and methods according to the disclosure also surprisingly reduces or eliminates the odor that is typically encountered with compositions and methods for removing color from hair.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the phrase “applying a composition onto keratin fibers” and variations thereof are intended to mean contacting the keratin fibers such as hair with at least one of the compositions of the disclosure, in any manner. It may also mean contacting the keratin fibers with an effective amount of the composition.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the terms “treat,” “treated,” “treatment,” and variations thereof is not intended to be limiting, but rather is merely intended to indicate that one or more compositions is applied to the hair, and optionally removed from the hair. For example, hair that is “treated” with a composition according to the disclosure may have had the composition applied, and/or may have had the composition applied and removed, e.g. by rinsing or towel drying. As a further example, hair that is “treated” with a composition according to the disclosure may have had the composition applied, and/or may have had the composition applied and rinsed from the hair.

For purposes of the disclosure, the terms “color removal,” “hair color-removing,” and variations thereof should be understood to refer to removal of artificial color from the hair imparted by previously-applied oxidative dyes. It should be understood that removal of color can include removal of all color, or can include removal of various tones of color. Therefore, in some embodiments, overall lightening of the hair color will be observed (e.g. greater ΔE), while in other embodiments even if the overall color change is not considered significant, variation in one or more of the L*, a*, or b* values will be observed.

The examples that follow serve to illustrate embodiments of the present disclosure without, however, being limiting in nature. It will be apparent to those skilled in the art that various modifications and variations can be made in the compositions and methods of the invention without departing from the spirit or scope of the invention.

### EXAMPLES

The following Examples are intended to be non-limiting and explanatory in nature only. In the Examples, amounts are expressed in percentage by weight (wt %) of active materials, relative to the total weight of the composition, unless otherwise indicated.

In these Examples, the change in the color of hair is evaluated with the CIE L* a* b* system, using Colorshot MS, where the change is determined by evaluating the color of the hair after treatment (L*₂, a*₂, b*₂) compared to the color of the hair before treatment (L*₁, a*₁, b*₁). The change in color (ΔE) is defined as:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

In this system, the three parameters represent, respectively, the color intensity (L*), the green/red color axis (a*) and the blue/yellow color axis (b*). The higher the value of L*, the lighter the color, the higher the value of a*, the redder the color, and the higher the value of b*, the yellower the color. The higher the value for ΔE, the greater the difference in color of treated hair relative to the color of the hair prior to treatment.

### Example 1—Compositions

Compositions 1A-1B according to the disclosure were prepared as shown in Table 1A, and comparative compositions C1-C3 were prepared as shown in Table 1B. Two commercially-available hair color remover compositions C4-C5 are shown in Table 1C, which shows the ingredients as listed on the packages.

TABLE 1A
Compositions according to the disclosure

| | 1A | 1B |
|---|---|---|
| Sodium metabisulfite | 10 | 5.0 |
| ß-cyclodextrin | 5.0 | 5.0 |
| Citric Acid | 0.50 | 0.50 |
| Fragrance | 1.0 | 1.0 |
| Xanthan Gum | 0.70 | 0.70 |
| Sodium Lauryl Sulfate | 1.5 | 1.5 |
| Water | QS to 100 | QS to 100 |

TABLE 1B
Comparative compositions

| | C1 | C2 | C3 |
|---|---|---|---|
| Sodium metabisulfite | 10 | 10 | 5.0 |
| Citric Acid | 0.50 | 0.50 | 0.50 |
| Fragrance | | 1.0 | 1.0 |
| Xanthan Gum | 0.70 | 0.70 | 0.70 |
| Sodium Lauryl Sulfate | 1.5 | 1.5 | 1.5 |
| Water | QS to 100 | QS to 100 | QS to 100 |

TABLE 1C
Commercial hair color-remover products

| | Ingredients listed on package |
|---|---|
| C4 | Sodium Persulfate, Potassium Persulfate, Sodium Lauryl Sulfate, |
| | Stronium Peroxide, Sodium Metasilicate, Ammonium Chloride, |
| | Cellulose Gum, Diethylhexyl Sodium Sulfosuccinate, EDTA, CI |
| | 77007/Ultramarines, Sodium Benzoate |
| C5 | Ascorbic Acid, Bentonite, Xanthan Gum, Disodium EDTA, |
| | Polyquaternium-10, Sodium Cocoyl Isethionate, Sodium Isethionate, |
| | Sodium Gluconate |

### Example 2—Evaluation of Color Removal

Seven swatches of virgin, ˜90% grey hair, and eight swatches of permed, ˜90% grey hair (15 swatches total) were colored with a commercially-available oxidative hair dye composition. The oxidative hair dye composition was mixed in a 1:1 ratio with 20V hydrogen peroxide developer to form a hair dye mixture, and the hair dye mixture was applied to the swatches at a rate of about 3 grams per gram of hair. The swatches were left to process at room temperature for about 35 minutes, rinsed, shampooed with a commercial shampoo, rinsed again, and allowed to air dry.

Once the swatches were dry, the swatches were treated as follows. Each of compositions 1A and C1-C5 were applied to one virgin swatch, and each of compositions 1A-1B and C1-C5 were applied to one permed swatch at a rate of about 2 grams per gram of hair, so that the color removing ability of each composition could be evaluated on both virgin hair colored with an oxidative dye and permed hair colored with an oxidative dye. The swatches were allowed to rest for 30 minutes at 33ºC. The swatches were then rinsed for about 30 seconds, shampooed with a commercial shampoo, rinsed, shampooed again, rinsed again, and then blow-dried.

One virgin and one permed swatch colored with the hair dye mixture were not treated with a color-removing composition, and were used as controls.

The color of each of the swatches was then evaluated with the CIE L* a* b* system.

### Example 2A—Evaluation of Color Removal Efficacy

The color of each of the swatches was evaluated immediately after the swatches were treated as described above and dried (time=T₀), to evaluate the effectiveness of color removal.

Table 2A-1 shows the L*, a, b* and ΔE values for swatches of virgin hair at T₀.

TABLE 2A-1

| | | L* | a* | b* | ΔE |
|---|---|---|---|---|---|
| L*₁, a*₁, b*₁ | CONTROL | 6.49 | 1.37 | 0.03 | — |
| L*₂, a*₂, b*₂ | 1A | 42.48 | 6.07 | 29.42 | 46.71 |
| | C1 | 32.17 | 6.98 | 26.04 | 36.98 |
| | C2 | 35.69 | 10.11 | 25.74 | 39.87 |
| | C3 | 21.59 | 4.90 | 5.12 | 16.32 |
| | C4 | 7.50 | 2.32 | 0.92 | 1.64 |
| | C5 | 12.41 | 13.39 | 7.15 | 15.17 |

Table 2A-2 shows the L*, a*, b* and ΔE values for swatches of permed hair at T₀.

TABLE 2A-2

| | | L* | a* | b* | ΔE |
|---|---|---|---|---|---|
| L*₁, a*₁, b*₁ | CONTROL | 5.90 | 1.64 | 0.20 | — |
| L*₂, a*₂, b*₂ | 1A | 48.55 | 6.84 | 18.39 | 46.65 |
| | 1B | 35.26 | 5.18 | 16.18 | 33.61 |
| | C1 | 43.16 | 8.54 | 17.87 | 41.81 |
| | C2 | 43.98 | 12.32 | 19.03 | 43.80 |
| | C3 | 36.26 | 5.51 | 14.20 | 33.65 |
| | C4 | 14.53 | 24.62 | 15.41 | 28.87 |
| | C5 | 14.92 | 22.50 | 13.76 | 26.46 |

Tables 2A-1 and 2A-2 demonstrate that compositions according to the disclosure are surprisingly more effective for removing color from hair, compared to compositions not according to the disclosure. This benefit is clear when considering the difference in overall hair color-removing results (ΔE) observed on the swatches treated with composition 1A compared to the results observed on the swatches treated with compositions C1-C2, which are identical except for the cyclodextrin and fragrance.

In addition, Tables 2A-1 and 2A-2 show that the compositions according to the disclosure perform significantly better than commercially-available color removal compositions C4 and C5, particularly on permed hair.

Since a difference in ΔE of ˜2 is visible to the naked eye, these results are significant.

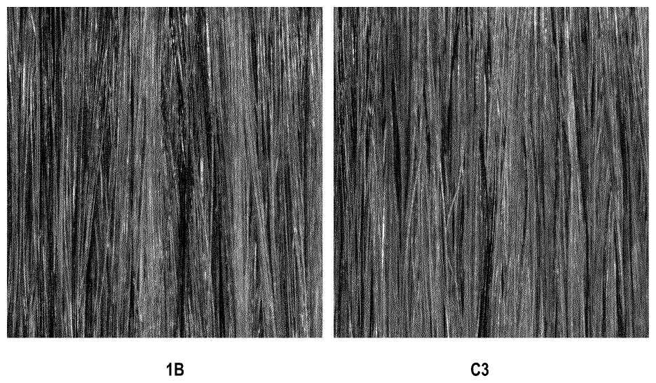

This Example also demonstrates that compositions according to the disclosure effectively alter the color, shade, or tone of the hair without having to remove all of the previously-applied color. In particular, Table 2A-2 shows differences in the L*, a*, and b* values of the swatch treated with composition 1B compared to the values of the swatch treated with composition C3, which are identical except for the cyclodextrin. The difference in b* values of the treated swatches demonstrates that the swatch treated with composition 1B has a warmer, more yellow undertone, showing that the blue tone was more effectively removed than in the swatch treated with composition C3, as seen in FIG. 1. This demonstrates that hair color-removing compositions according to the disclosure can also be effectively used to adjust the color or tone of the hair.

### Example 2B—Evaluation of Lastingness of Color Removal

The color of the swatches treated with compositions 1A and C1-C3 was again evaluated after one week (time=T₇), to evaluate the lastingness of the color removal.

Table 2B-1 shows the L*, a*, b* and ΔE values for swatches of virgin hair at T₇.

TABLE 2B-1

| | | L* | a* | b* | ΔE |
|---|---|---|---|---|---|
| L*₁, a*₁, b*₁ | CONTROL | 6.49 | 1.37 | 0.03 | — |
| L*₂, a*₂, b*₂ | 1A | 45.13 | 7.06 | 29.53 | 48.94 |
| | C1 | 37.13 | 8.07 | 29.82 | 43.26 |
| | C2 | 36.17 | 10.80 | 26.76 | 41.04 |
| | C3 | 37.82 | 6.56 | 25.73 | 40.85 |

Table 2B-2 shows the L*, a*, b* and ΔE values for swatches of permed hair at T₇.

TABLE 2B-2

| | | L* | a* | b* | ΔE |
|---|---|---|---|---|---|
| L*₁, a*₁, b*₁ | CONTROL | 5.90 | 1.64 | 0.20 | — |
| L*₂, a*₂, b*₂ | 1A | 51.46 | 6.97 | 19.45 | 49.75 |
| | C1 | 43.42 | 9.47 | 18.79 | 42.59 |
| | C2 | 46.31 | 12.78 | 18.32 | 45.66 |
| | C3 | 43.29 | 7.24 | 20.09 | 42.71 |

Although the L*, a*, b* and ΔE values for the swatches treated with composition 1A are slightly higher or lower in Tables 2B-1 and 2B-2 than in Tables 2A-1 and 2A-2, these differences are considered to be within an acceptable deviation. Visually, there was no difference in color of the swatch at T₇ compared to T₀.

Tables 2B-1 and 2B-2 therefore demonstrate that the color removal lasts through at least one week. It was observed that even after several months, the swatches treated with composition 1A visually appeared to maintain the same level of color removal, demonstrating that re-oxidation of the color does not occur.

### Example 3—Evaluation of Odor Control

Consistent with how such evaluations are typically done, performance of odor control was evaluated by a fragrance expert, who reported the results shown in Table 3 for the odor detected for compositions 1A, C1, and C2, as well as for the swatches treated with each of compositions 1A, C1, and C2.

TABLE 3

| | Composition odor | Hair swatch odor |
|---|---|---|
| C1 | Sharp vinegar odor | Odor remains on swatch |
| C2 | Strong floral scent, but base odor | Fragrance detected on swatch |
| | still present | |
| 1A | Best coverage, some base odor still | Odor covered more on swatch |
| | present | |

The expert confirmed that composition 1A had a less-offensive odor than compositions C1 and C2, and that hair treated with composition 1A had a less-offensive odor than hair treated with compositions C1 and C2. Thus, Example 3 demonstrates that compositions according to the disclosure have surprisingly better odor control than compositions not according to the disclosure.

### Example 4—Additional Compositions

Compositions 4A-4F according to the disclosure can be prepared as shown in Table 4, and are likewise expected to provide synergistic hair color removing and reduced odor benefits.

TABLE 4

| | 4A | 4B | 4C | 4D | 4E | 4F |
|---|---|---|---|---|---|---|
| Sodium bisulfite | 13 | 6 | | | 1 | |
| Potassium | | 4 | 4 | | | |
| metabisulfite | | | | | | |
| Ammonium sulfite | | | 2 | 3 | | |
| Thiolactic acid | | | | | 2 | |
| Thioglycolic acid | | | | | | 15 |
| β-cyclodextrin | 11 | | 6 | 3 | | 15 |
| α-cyclodextrin | | 8 | | | 5 | |
| Citric Acid | 0.4 | 0.5 | 0.6 | 0.5 | 0.3 | 0.7 |
| Hydroxypropyl guar | 0.5 | 0.7 | 1 | 0.7 | 1.5 | 2 |
| Sodium Lauroyl | 2 | 1.5 | 1 | 1.5 | 2 | 1 |
| Sulfate | | | | | | |
| Water | QS to | QS to | QS to | QS to | QS to | QS to |
| | 100 | 100 | 100 | 100 | 100 | 100 |

The above Examples demonstrate that compositions and methods according to the disclosure, which combine reducing agents and cyclodextrin compounds to remove color from hair, surprisingly and unexpectedly enhance removal of oxidative hair dyes from hair, which removal is long-lasting, compared to compositions and methods not according to the disclosure. The Examples further demonstrate that improved odor control of hair-color removing compositions is also achieved by compositions according to the disclosure.

1. A hair-color removing composition comprising:
a) at least one reducing agent chosen from sulfur-based reducing agents, thio-based reducing agents, salts thereof, or combinations of two or more thereof;
b) at least one cyclodextrin compound; and
c) at least one solvent.

2. The composition of claim 1, comprising at least one reducing agent chosen from sulfites, bisulfites, salts thereof, or combinations of two or more thereof.

3. The hair-color removing composition of claim 1, comprising at least one reducing agent chosen from thiolactic acid, thioglycolic acid, sodium sulfite, potassium sulfite, ammonium sulfite, sodium bisulfite, ammonium bisulfite, sodium metabisulfite, potassium metabisulfite, or combinations of two or more thereof.

4. The hair-color removing composition of claim 1, wherein the total amount of reducing agents ranges from about 1% to about 15% by weight, relative to the total weight of the composition.

5. The hair-color removing composition of claim 1, comprising β-cyclodextrin.

6. The hair-color removing composition of claim 1, wherein the total amount of cyclodextrin compounds ranges from about 1% to about 15% by weight, relative to the total weight of the composition.

7. The hair-color removing composition of claim 1, wherein the weight ratio of the total amount of reducing agents to the total amount of cyclodextrin compounds ranges from about 1 to about 5.

8. The hair-color removing composition of claim 1, further comprising at least one surfactant chosen from anionic surfactants, non-ionic surfactants, amphoteric/zwitterionic surfactants, or combinations of two or more thereof.

9. A hair-color removing composition comprising:
a) at least one reducing agent;
b) at least one cyclodextrin compound; and
c) water,
wherein the weight ratio of the total amount of reducing agents to the total amount of cyclodextrin compounds ranges from about 1 to about 5.

10. The composition of claim 9, comprising at least one reducing agent chosen from sulfites, bisulfites, salts thereof, or combinations of two or more thereof.

11. The hair-color removing composition of claim 9, comprising at least one reducing agent chosen from thiolactic acid, thioglycolic acid, sodium sulfite, potassium sulfite, ammonium sulfite, sodium bisulfite, ammonium bisulfite, sodium metabisulfite, potassium metabisulfite, or combinations of two or more thereof.

12. The hair-color removing composition of claim 9, wherein the total amount of reducing agents ranges from about 0.1% to about 20% by weight, relative to the total weight of the composition.

13. The hair-color removing composition of claim 9, comprising β-cyclodextrin.

14. The hair-color removing composition of claim 9, wherein the total amount of cyclodextrin compounds ranges from about 0.1% to about 20% by weight, relative to the total weight of the composition.

15. The hair-color removing composition of claim 9, further comprising at least one surfactant chosen from anionic surfactants, non-ionic surfactants, amphoteric/zwitterionic surfactants, or combinations of two or more thereof.

16. A method for removing color from hair, the method comprising:
i. applying to the hair a hair-color removing composition comprising:
a. at least one reducing agent chosen from sulfur-based reducing agents, thio-based reducing agents, salts thereof, or combinations of two or more thereof;
b. at least one cyclodextrin compound; and
c. at least one solvent, and
ii. rinsing the hair.

17. The method according to claim 16, wherein the hair-color removing composition comprises at least one reducing agent chosen from thiolactic acid, thioglycolic acid, sodium sulfite, potassium sulfite, ammonium sulfite, sodium bisulfite, ammonium bisulfite, sodium metabisulfite, potassium metabisulfite, or combinations of two or more thereof.

18. The method according to claim 16, wherein the hair-color removing composition comprises a total amount of reducing agents and a total amount of cyclodextrin compounds in a weight ratio of reducing agents to cyclodextrin compounds ranging from about 1 to about 5.

19. The method according to claim 16, wherein the hair-color removing composition is left on the hair for a period of time ranging from about 1 minute to about 60 minutes before the hair is rinsed.

20. The method according to claim 16, wherein the hair to which the hair-color removing composition is applied comprises at least one oxidative dye.
